(19) United States
(12) Patent Application Publication
(10) Pub. No.: US 2022/0175847 A1
(43) Pub. Date: Jun. 9, 2022

(54) Salivary Gland Cell Sheets and Methods for their Production and Use

(71) Applicant: University of Utah Research Foundation, Salt Lake City, UT (US)

(72) Inventors: Olga Baker, Salt Lake City, UT (US); Teruo Okano, Salt Lake City, UT (US); Kihoon Nam, Salt Lake City, UT (US); Kyungsook Kim, Murray, UT (US)

(73) Assignee: University of Utah Research Foundation, Salt Lake City, UT (US)

(21) Appl. No.: 17/435,885

(22) PCT Filed: Mar. 5, 2020
(86) PCT No.: PCT/US2020/021082

§ 371 Date: Sep. 2, 2021

Related U.S. Application Data
(60) Provisional application No. 62/815,079, filed on Mar. 7, 2019.

(51) Int. Cl. A61K 35/38; C12N 5/071

(52) U.S. Cl. CPC A61K 35/38; C12N 5/0633

(57) ABSTRACT

The disclosure provides a salivary gland (SG) cell sheet comprising one or more layers of confluent SG cells (e.g. submandibular gland (SMG) cells). Methods of treating a wound in a salivary gland (SG), treating hyposalivation, and treating irradiation damage in an SG are also provided. The disclosure also provides a method for producing SG cell sheets comprising culturing SG cells in culture solution on a temperature-responsive polymer which has been coated onto a substrate surface of a cell culture support, wherein the temperature-responsive polymer has a lower critical solution temperature in water of 0-80° C.; adjusting the temperature of el the culture solution to below the lower critical solution temperature, whereby the substrate surface is made hydrophilic and adhesion of the cell sheet to the surface is weakened; and detaching the cell sheet from the culture support.

### RELATED APPLICATIONS

This application claims priority to U.S. Provisional Patent Application No. 62/815,079 filed on Mar. 7, 2019, the contents of which are incorporated herein in their entirety.

### BACKGROUND OF THE INVENTION

Progress to cure hyposalivation is slow with current treatments limited to saliva substitutes (which provide only temporary relief) and secretory agonists (for which secondary effects include sweating, nausea, dizziness, weakness, diarrhea, and blurred vision). See Dost et al., Aust Dent J. 2013, 58:11-7; Porter et al., Oral Surg Oral Med Oral Pathol Oral Radiol Endod. 2004, 97:28-46; Villa et al., Therapeutics and clinical risk management, 2014, 11:45-51; Braga et al., Int J Dent Hyg. 2009, 7:126-30; Fox et al., Oral Surg Oral Med Oral Pathol. 1992; 74:315-8; Martinez et al., J Dent Res. 1983; 62:543-7; Rhodus et al., Oral Dis. 1997; 3:93-8; Rhodus et al., Oral Surg Oral Med Oral Pathol. 1991; 72:545-9. Several alternative therapies including the use of stem cells as well as various scaffolds are additional options for boosting secretory function. Regarding stem cells, recent studies have demonstrated that their transplantation restores saliva secretion and tissue homeostasis in irradiated glands. See Nanduri et al., Radiother Oncol. 2011; 99:367-72; Nanduri et al., Radiother Oncol. 2013; 108:458-63. However, further studies are needed to determine both how to incorporate stem cells into host tissues and what secondary effects might arise. Regarding use of scaffolds, a variety of nanofibers, polysaccharides and PEGT/PBT have been shown to promote growth, cell attachment, migration and proliferation; however, the level of structural organization (as demonstrated by poor hollow multi-lumen formation, impaired 3D cell polarity and limited functionality) has been modest. See Cantara et al., Biomaterials. 2012; 33:8372-82; Soscia et al., Biomaterials. 2013; 34:6773-84; Hsiao et al., Data Brief. 2015; 4:551-8; Yang et al., Biomaterials. 2015; 66:29-40; Sun et al, Archives of oral biology. 2006; 51:351-8. Additionally, studies have shown that human cells grown on a hyaluronic acid based scaffold and transplanted into a wounded glands allowed integration of the scaffold into the healing area with some regeneration markers noted (Pradhan-Bhatt et al., Laryngoscope 2014; 124:456-61); however, scaffold stability must be determined and cell tracking performed to establish the utility of this procedure. Thus a need exists for improved methods of treating hyposalivation and other disorders of the salivary gland.

### SUMMARY OF THE INVENTION

In certain aspects the disclosure relates to a salivary gland (SG) cell sheet comprising one or more layers of confluent SG cells. In certain embodiments, the SG cells are arranged in a columnar pattern with a flat basolateral side and a protrusive apical side. In certain embodiments, the cell sheet comprises one or more intercellular junctions selected from the group consisting of tight junctions (TJ), adherens junctions (AJ), and desmosomes (DS). In certain embodiments, at least 50% of the SG cells in the cell sheet are connected by an intercellular junction selected from the group consisting of tight junctions (TJ), adherens junctions (AJ) and desmosomes (DS). In certain embodiments, the cell sheet comprises one or more of microvilli-like structures and secretory granules. In certain embodiments, at least 50% of the SMG cells in the cell sheet comprise microvilli-like structures or secretory granules. In certain embodiments, the SMG cells express one or more proteins selected from the group consisting of tight junction protein zonula occludens-1 (ZO-1), E-cadherin, aquaporin 5 (AQP5) and F-actin. In certain embodiments, the cell sheet consists essentially of SG cells. In certain embodiments, at least 50% of cells in the cell sheet are SG cells. In certain embodiments, the SG cells are human SG cells.

In certain aspects the disclosure relates to a composition comprising an SG cell sheet as described herein and a polymer-coated culture support that is removable from the cell sheet. In certain aspects the disclosure relates to a composition comprising at least two of the SG cell sheets described herein. In certain embodiments, the at least two cell sheets are stacked on top of each other. In certain embodiments, the cell sheets comprise epithelial lumens. In certain embodiments, the SG cells are submandibular gland (SMG) cells.

In certain aspects the disclosure relates to a method of treating a wound in a salivary gland (SG), the method comprising applying an SG cell sheet as described herein or a composition comprising the cell sheet as described herein to a wounded SG.

In certain embodiments, applying the cell sheet or the composition to the SG results in wound closure of at least 50%. In certain embodiments, applying the cell sheet or the composition to the wounded SG increases expression of one or more proteins selected from the group consisting of zonula occludens-1 (ZO-1), E-cadherin, aquaporin 5 (AQP5), cytokeratin 7, transmembrane protein 16 (TMEM16), and sodium potassium ATPase (Na⁺/K⁺-ATPase) in the wounded SMG relative to a wounded SMG to which the cell sheet or composition is not applied. In certain aspects the disclosure relates to a method of treating hyposalivation in a subject, the method comprising applying an SG cell as described herein or a composition comprising the cell sheet as described herein to a salivary gland (SG) in a subject. In certain embodiments, applying the cell sheet or the composition to the SG increases saliva flow rate in the subject relative to a subject in which the cell sheet or composition is not applied. In certain embodiments, the saliva flow rate is increased by at least 50% in the subject to which the cell sheet or composition is applied relative to a subject in which the cell sheet or composition is not applied.

In certain aspects the disclosure relates to a method of treating irradiation damage in a salivary gland (SG), the method comprising applying an SG cell sheet or composition comprising the cell sheet as described herein to a SG that has been damaged by irradiation. In certain embodiments, the SG cells are autologous to the subject. In certain embodiments, the SG cells are allogeneic to the subject. In certain embodiments, the subject is a human. In certain embodiments, the SG is a submandibular gland (SMG) and the SG cells are SMG cells.

In certain aspects the disclosure relates to a method for producing a salivary gland (SG) cell sheet comprising one or more layers of confluent SG cells, the method comprising: a) culturing SG cells in culture solution on a temperature-responsive polymer which has been coated onto a substrate surface of a cell culture support, wherein the temperature-responsive polymer has a lower critical solution temperature in water of 0-80° C.; b) adjusting the temperature of the culture solution to below the lower critical solution temperature, whereby the substrate surface is made hydrophilic and adhesion of the cell sheet to the surface is weakened; and c) detaching the cell sheet from the culture support.

In certain embodiments, the culture solution comprises one or more components selected from the group consisting of DMEM/F12 complete medium, fetal bovine serum (FBS), triiodothyronine, retinoic acid, hydrocortisone, epidermal growth factor (EGF), sodium selenite, glutamine, insulin and transferrin. In certain embodiments, the adjusting step (b) is performed when the SG cells are confluent. In certain embodiments, the culturing step (a) comprises adding the SG cells to the culture solution at an initial cell density of at least 1×10⁵ cells/cm². In certain embodiments, the SG cells are cultured in the culture solution on the temperature-responsive polymer for at least 8 days before the adjusting step (b). In certain embodiments, the SG cells are submandibular gland (SMG) cells. In certain aspects the disclosure relates to a cell sheet produced by any of the methods described herein.

### BRIEF DESCRIPTION OF THE DRAWINGS

FIGS. 1A, 1B and 1C show that submandibular gland (SMG) cells form single sheets. (A) SMG tissue was dissociated using a GentleMACS and plated on a thermoresponsive culture dish at 37° C. for eight days, as described in Materials and Methods. Dish temperature was then reduced to 25° C., which in turn caused the cells to detach from the surface and subsequently maintained intact the extracellular matrix proteins as compared to a traditional cell isolation method using trypsin, in which these proteins quickly disperse. (B) Sequence of cells detaching from the thermoresponsive plate depicted at 3, 5 and 8 min, with complete detachment occurring after 30 min. White bars represent 200 μm. (C) Single layer cell sheets were embedded in paraffin, sectioned, stained with H&E and imaged using a Leica DMI6000B inverted microscope at 10×. Black bars represent 100 μm.

FIGS. 2A, 2B and 2C show that submandibular gland-derived single layer cell sheets maintain tight junctions and secretory granules. Shown are transmission electron micrographs (TEM) of submandibular gland cells grown on thermoresponsive plates for 8 days. Cells were processed for morphological analysis, as described in Materials and Methods and cell junctions (A) and secretory granules (B) detected and compared to native submandibular gland (C). Data are representative of results from 3 or more experiments. Microvilli (Mi), Tight Junction (TJ), Adherens Junction (AJ), Desmosomes (DS), Secretory Granules (SG).

FIG. 3A-3F show that single layer cell sheets polarize and differentiate while double layer cell sheets form a glandular-like appearance. Submandibular gland single or double layer cell sheets were imaged using a differential interference contrast microscopy (A, D) scale bars=200 μm as well as using a Carl Zeiss 710 confocal microscope at 40× (scale bars=50 μm) with the following specifications rabbit anti-ZO-1 (B and E) and mouse anti-E-cadherin (B and E), rabbit anti-aquaporin 5 (C and F) and F-actin (C and F). Data are representative of results from 3 or more experiments.

FIG. 4A-4H show that double layer cell sheets can be directly transplanted to wounded submandibular glands. Skin incisions of approximately 1 cm in length were made along the anterior surface of the neck of a C57BL/6J and SMG were exposed (A-C). Then, a 3 mm diameter biopsy punch was performed and (D) surgical wounds filled with a single or double layer cell sheet measuring approximately 1 cm of diameter of a semicircle (E-G). Finally, the skin incision was sutured and mice were placed in a recovery room (H).

FIG. 5A-5H show that double layer cell sheets promote tissue organization to a similar extent as sham controls at post-surgery day 8. H&E staining of wounded SMG that (A; day 8 and E; day 20) remained untreated, (B; day 8 and F; day 20) were treated with single layer cell sheets (C; day 8 and G; day 20) were treated with double layer cell sheets or (D; day 8 and H; day 20) were unwounded (sham controls) was performed and specimens were analyzed by light microscopy using a Leica DMI6000B as described in Materials and Methods; bars=1 mm with dotted areas indicating wounded areas. Data are representative of results from 3 or more experiments.

FIG. 6A-6J show that double layer cell sheets promote epithelial polarity to a similar extent as sham controls. Confocal analysis of wounded SMG that remained untreated (A; day 8 and E; day 20), were treated with single layer cell sheets (B; day 8 and F day; 20), were treated with double layer cell sheets (C; day 8 and G; day 20) or were unwounded (sham controls) (D; day 8 and H; day 20) was performed as follows: rabbit anti-ZO-1 and mouse anti-E-cadherin. Data are representative of results from 5 experiments. White bars represent 100 μm. Positive area of ZO-1 (I) and E-cadherin (J) was calculated using ImageJ and analyzed using one-way ANOVA (*p<0.05, n=5, ns=not significant) with Tukey's multiple comparisons to the Sham control (day 8).

FIG. 7A-7J show that double layer cell sheets (DC) promote aquaporin-5 expression to a similar extent as sham controls. Confocal analysis of wounded SMG that remained untreated (A; day 8 and E; day 20), were treated with single layer cell sheets (SC) (B; day 8 and F day; 20), were treated with double layer cell sheets (C; day 8 and G; day 20) or were unwounded (sham controls) (D; day 8 and H; day 20) was performed as follows: anti-aquaporin 5 (green) and mouse anti-cytokeratin 7. Data are representative of results from 5 experiments. White bars represent 100 μm. Positive area of aquaporin 5 (I) and cytokeratin (J) was calculated using ImageJ and analyzed using one-way ANOVA (*p<0.05, n=5, ns=not significant) with Tukey's multiple comparisons to the Sham control (day 8).

FIG. 8A-8J show that double layer cell sheets (DC) promote TMEM16 expression. Confocal analysis of wounded SMG that remained untreated (A; day 8 and E; day 20), were treated with single layer cell sheets (SC) (B; day 8 and F day; 20), were treated with double layer cell sheets (C; day 8 and G; day 20) or were unwounded (sham controls) (D; day 8 and H; day 20) was performed as follows: rabbit anti-TMEM16A and mouse anti-Na⁺/K⁺ ATPase. Data are representative of results from 5 experiments. White bars represent 100 μm. Positive area of TMEM16A (I) and Na+/K+ ATPase (J) was calculated using ImageJ and analyzed using one-way ANOVA (*p<0.05, n=5, ns=not significant) with Tukey's multiple comparisons to the Sham control (day 8).

FIGS. 9A and 9B show that double layer cell sheets restore body weight and saliva secretion to a similar extent as sham controls. (A) After submandibular gland wounds were made, mice received the indicated treatments and changes in body weight (%) of untreated (●), single (▪) or double layer cell sheet (▴) treated mice groups were compared with sham control group (o) over 20-day period. Data represent the means±SD of n=9 mice per condition where statistical significance was assessed by two-way ANOVA (P<0.05) and Dunnett's post-hoc test for multiple comparisons to the sham group. (B) After the various treatments, mice were anesthetized and stimulated with pilocarpine and isoproterenol as described in Example 2. Then, saliva was collected for 5 min. Untreated, wounded (Control), wounded treated with single layer cell sheets (SC), wounded treated with double layer cell sheets (DC) were compare to the sham (unwounded) control groups (Sham). Data represent the means±SD of n=7 mice per condition and statistical significance was assessed by two-way ANOVA (P<0.01) and Tukey's multiple comparisons to the Sham control (day 8).

FIG. 10 shows that a cell sheet applied to a mouse submandibular gland (SMG) restores saliva composition. Untreated, wounded (Control), wounded treated with single layer cell sheets (SC), wounded treated with double layer cell sheets (DC) were compare to the sham (unwounded) control groups (Sham).

FIG. 11 shows that human submandibular gland (SMG) cells form single sheets. Human SMG tissue (about 100 mg) was dissociated using a GentleMACS and plated on a thermoresponsive culture dish at 37° C. for eight days, as described in Materials and Methods. Dish temperature was then reduced to 25° C. Single layer cell sheets were embedded in paraffin, sectioned, stained with H&E and imaged using a Leica DMI6000B inverted microscope at 10×. Black bars represent 100 mm or 200 μm.

### DETAILED DESCRIPTION OF THE INVENTION

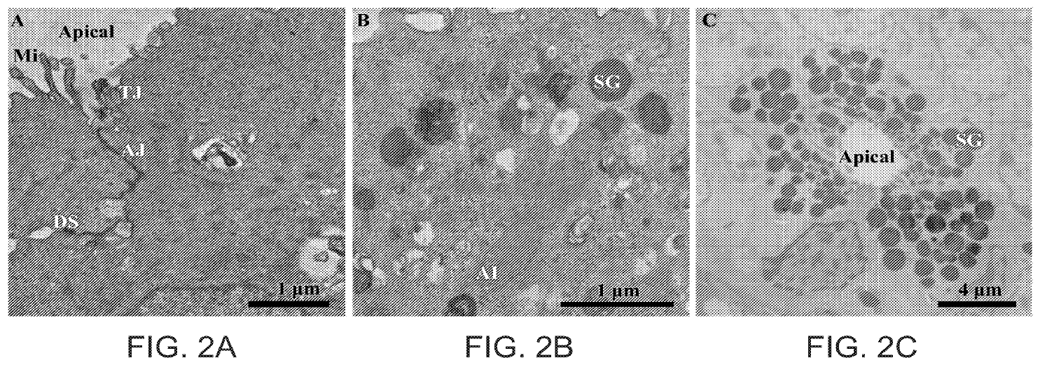

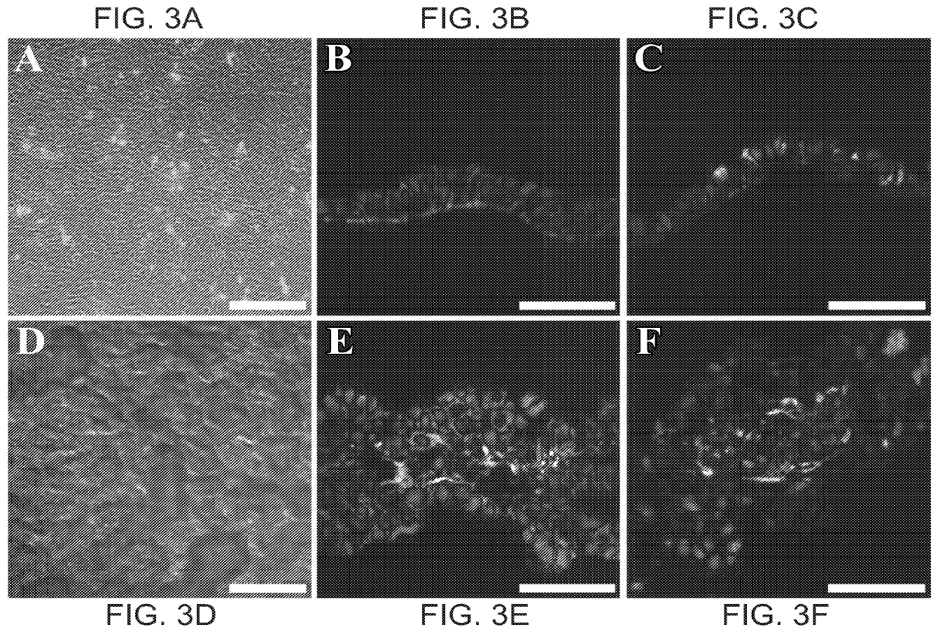

This disclosure describes preparation and properties of salivary gland (SG) cell sheets and their use for treating disorders of the SG such as wounding of the SG, hyposalivation, and irradiation damage of the SG. In a particular embodiment, the SG cell is a submandibular gland (SMG) cell. SMG cells were used to prepare cell sheets in vitro in temperature-responsive cell culture dishes (TRCDs) coated with a temperature-responsive polymer. Confluent cell sheets formed at 8 days after seeding and were detached from the TRCD by cooling the cultures to room temperature. As described herein, some of the advantages of SMG cell sheets include maintenance of intrinsic extracellular matrix (ECM) proteins, tight junctions (TJs) and secretory granules (FIG. 2). Additionally, double layer SMG cell sheets are able to form acinar and ductal-like organoids with a three-dimensional shape containing lumens and consistent with salivary gland epithelium (FIG. 3). These features offer an improvement over freshly isolated SMG cells plated on plastic where they lose secretory granules, form disorganized TJs and de-differentiate over time (Redman et al., Biotech Histochem. 2008; 83:103-30). In addition, application of the cell sheets to a surgically wounded submandibular gland (SMG) in mice improved wound closure, and increased body weight and saliva flow rates.

I. Submandibular Gland (SMG) Cell

The term “salivary gland cell” includes but is not limited to cells of the submandibular gland (SMG), sublingual gland, and parotid gland.

The term “submandibular gland cell” or “SMG” includes but is not limited to acinar cells, ductal cells, serous cells, mucous cells, myoepithelial cells, nerves, stem cells and progenitor cells. In some embodiments the concentration of acinar cells, ductal cells, serous cells, mucous cells, myoepithelial cells, nerves, stem cells or progenitor cells in the cell sheet is at least 1%, 2%, 3%, 4%, 5%, 10%, 20%, 30%, 40%, 50%, 60%, 70%, 80%, 90%, 95% or 99%. In some embodiments the concentration of acinar cells, ductal cells, serous cells, mucous cells, myoepithelial cells, nerves, stem cells or progenitor cells in the cell sheet is less than 1%, 2%, 3%, 4%, 5%, 10%, 20%, 30%, 40%, 50%, 60%, 70%, 80%, 90%, 95% or 99%. Any of these values may be used to define a range for the concentration of each type of cell in the cell sheet. For example, in some embodiments, the concentration of acinar cells in the cell sheet is from 5% to 50% of the cells in the cell sheet.

Methods for isolating SMG cells from SMGs are known in the art and are described, for example, in US 2017/0056557, which is incorporated by reference herein in its entirety.

The SG cell sheets described herein differ from SG cell suspension cultures in several ways. Suspension cultures of SG cells contain single cells that do not have an ECM or cell-cell junctions because the adhesive proteins in these cell-cell junctions must be removed (e.g. by trypsin treatment) to harvest cells from culture surfaces for preparation of the cell suspension culture. In contrast to singe cell suspensions of SG cells, the SG cell sheets described herein contain both an ECM and cell-cell junctions among the SG cells that are generated during formation of the cell sheet. The ECM and cell-cell junctions facilitate adhesion of the SG cell sheet to target tissue during transplantation to a host organism.

II. Cell Sheets Produced from Salivary Gland (SG) Cells

In certain aspects the present disclosure relates to a salivary gland (SG) cell sheet comprising one or more layers of confluent SG cells. The term “salivary gland cell sheet” or “SG cell sheet” as used herein refers to a cell sheet obtained by growing SG cells (e.g. SMG cells) on a cell culture support in vitro. The SG sheets described herein are harvested as a single sheet with a temperature shift using a temperature-responsive culture dish (TRCD) without any enzyme treatment. Accordingly, in certain aspects, the present disclosure relates to a composition comprising an SG cell sheet as described herein and a polymer-coated culture support (e.g. a culture dish) that is removable from the cell sheet.

The SG cell sheets maintain their shape by retaining tissue-like structures, actin filaments, extracellular matrix, intercellular proteins, and high cell viability, all of which are related to improved cell survival and cellular function. Accordingly, the cell sheets described herein may comprise structural features that improve cell survival and cell function, including an extracellular matrix, cell adhesion proteins and cell junction proteins. Thus, the SG cell sheets prepared by the methods described herein have several beneficial characteristics compared to SMG cell compositions produced by other methods. For example, chemical disruption (proteolytic enzyme treatment) may be used in preparation of suspension cultures of SG cells. However, the chemical disruption method is unable to maintain tissue-like structures of cells as well as cell-cell communication, since enzyme treatment disrupts the extracellular and intracellular proteins (cell-cell and cell-ECM junctions). Accordingly, protein cleavage by enzymes reduces cell viability and cellular functions.

In some embodiments, the SG cells (e.g. SMG cells) are arranged in a columnar pattern with a flat basolateral side and a protrusive apical side. The term “basolateral” as used herein refers to the membrane above the tight junctions between SG cells that faces the plasma. The term “apical” as used herein refers to the membrane below the tight junction facing the lumen.

The SG cell sheet may comprises one or more intercellular junctions selected from the group consisting of tight junctions (TJ), adherens junctions (AJ), and desmosomes (DS). In some embodiments, at least 10%, 20%, 30%, 40%, 50%, 60%, 70%, 80%, 90%, 95% or 99% of the SG cells (e.g. SMG cells) in the cell sheet are connected by an intercellular junction selected from the group consisting of tight junctions (TJ), adherens junctions (AJ) and desmosomes (DS). Tight junctions are multiprotein junctional complexes whose general function is to prevent leakage of transported solutes and water and seal the paracellular pathway. Adherens junctions are protein complexes that occur at cell-cell junctions in epithelial and endothelial tissues, and whose cytoplasmic face is linked to the actin cytoskeleton. They can appear as bands encircling the cell or as spots of attachment to the extracellular matrix. Desmosomes are spot-like cell structures specialized for cell-to-cell adhesion randomly arranged on the lateral sides of plasma membranes.

The SG cell sheet may also comprise additional structural features such as one or more of microvilli-like structures and secretory granules. Microvilli are cell membrane protrusions involved in a wide variety of cell functions, including absorption, secretion, and cellular adhesion. Secretory granules are small intracellular structures that contain specific proteins and other macromolecules destined for secretion into the extracellular space. In some embodiments, at least 10%, 20%, 30%, 40%, 50%, 60%, 70%, 80%, 90%, 95% or 99% of the SG cells in the cell sheet comprise microvilli-like structures and/or secretory granules.

The SG cells in the cell sheet may express one or more proteins selected from the group consisting of tight junction protein zonula occludens-1 (ZO-1), E-cadherin, aquaporin 5 (AQP5) and F-actin.

During isolation of salivary gland cells, other additional cells outside of the salivary gland may also be isolated. These additional cells include but are not limited to myoepithelial cells, progenitor cells, stem cells, endothelial cells, and nerves cells. In some embodiments, the cell sheet consists of or consists essentially of SG cells (e.g. SMG cells). In some embodiments, at least 10%, 20%, 30%, 40%, 50%, 60%, 70%, 80%, 90%, 95% or 99% of cells in the cell sheet are SG cells (e.g. SMG cells). In a particular embodiment, the SG cells in the cell sheet are human SG cells (e.g. human SMG cells).

Applicants have found that the combination of two single layer SMG cell sheets as described herein promotes formation of a glandular-like appearance tissue in vitro. For example, as describes in Example 1 and shown in FIG. 3D-F, placing two single cell sheets on top of each other for one day resulted in the formation of a double layer cell sheet with a glandular-like appearance where the majority of cells displayed organized round structures consistent with epithelial lumens. Accordingly, in some embodiments, the present disclosure relates to a composition comprising 2, 3, 4, 5, 6, 7, 8, 9, 10 or more SG cell sheets. The cell sheets may be stacked on top of each other for at least 1, 2, 3, 4, 5, 6, 7, 8, 9, 10 or more days to allow for the formation of organized structures such as epithelial lumens before transferring the cell sheets to an organism.

III. Methods for Producing SG Cell Sheets In Vitro

In certain aspects, the present disclosure relates to a method for producing a salivary gland (SG) cell (e.g. a SMG cell) sheet comprising one or more layers of confluent SG cells, the method comprising:
a) culturing SGs in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the culture support.

Methods for isolating SMG cells are known in the art and are described, for example, in US 2017/0056557, which is incorporated by reference herein in its entirety. For example, SMG tissue may be cut into small pieces and placed in culture medium containing tumor dissociation enzyme mixture. The tissue may be dissociated, for example by using a GentleMACS dissociator, and incubated in a shaking water bath at 37° C. Multiple rounds of dissociation and incubation may be performed, followed by centrifugation to remove the culture medium. The cells may then be resuspended in fresh culture solution and strained before seeding onto TRCDs for formation of the SMG cell sheets. In some embodiments, the culture solution comprises one or more components selected from the group consisting of DMEM/F12 complete medium, fetal bovine serum (FBS), triiodothyronine, retinoic acid, hydrocortisone, epidermal growth factor (EGF), sodium selenite, glutamine, insulin and transferrin. In a particular embodiment, the culture solution comprises DMEM/F12 complete medium, fetal bovine serum (FBS), triiodothyronine, retinoic acid, hydrocortisone, epidermal growth factor (EGF), sodium selenite, glutamine, insulin and transferrin.

General methods for preparing cell sheets are known in the art and are described, for example, in U.S. Pat. Nos. 8,642,338; 8,889,417; 9,981,064; and 9,114,192, each of which is incorporated by reference herein in its entirety.

The temperature-responsive polymer used to coat the substrate of the cell culture support has an upper or lower critical solution temperature in aqueous solution which is generally in the range of 0° C. to 80° C., for example, 10° C. to 50° C., 0° C. to 50° C., or 20° C. to 45° C.

The temperature-responsive polymer may be a homopolymer or a copolymer. Exemplary polymers are described, for example, in Japanese Patent Laid-Open No. 211865/1990. Specifically, they may be obtained by homo- or co-polymerization of monomers such as, for example, (meth)acrylamide compounds ((meth)acrylamide refers to both acrylamide and methacrylamide), N-(or N,N-di)alkyl-substituted (meth)acrylamide derivatives, and vinyl ether derivatives. In the case of copolymers, any two or more monomers, such as the monomers described above, may be employed. Further, those monomers may be copolymerized with other monomers, one polymer may be grafted to another, two polymers may be copolymerized, or a mixture of polymer and copolymer may be employed. If desired, polymers may be crosslinked to an extent that will not impair their inherent properties.

The substrate which is coated with the polymer may be of any types including those which are commonly used in cell culture, such as glass, modified glass, polystyrene, poly(methyl methacrylate), and ceramics.

Methods of coating the support with the temperature-responsive polymer are known in the art and are described, for example, in Japanese Patent Laid-Open No. 211865/1990. Specifically, such coating can be achieved by subjecting the substrate and the above-mentioned monomer or polymer to, for example, electron beam (EB) exposure, irradiation with γ-rays, irradiation with UV rays, plasma treatment, corona treatment, or organic polymerization reaction. Other techniques such as physical adsorption as achieved by coating application and kneading may also be used.

The coverage of the temperature responsive polymer may be in the range of 0.4-3.0 μg/cm², for example, 0.7-2.8 μg/cm², or 0.9-2.5 μg/cm². The morphology of the cell culture support may be, for example, a dish, a multi-plate, a flask or a cell insert.

The cultured cells may be detached and recovered from the cell culture support by adjusting the temperature of the support material to the temperature at which the polymer on the support substrate hydrates, whereupon the cells can be detached. Smooth detachment can be realized by applying a water stream to the gap between the cell sheet and the support. Detachment of the cell sheet may be affected within the culture solution in which the cells have been cultivated or in other isotonic fluids, whichever is suitable.

In a particular embodiment, the temperature-responsive polymer is poly(N-isopropyl acrylamide) Poly(N-isopropyl acrylamide) has a lower critical solution temperature in water of 31° C. If it is in a free state, it undergoes dehydration in water at temperatures above 31° C. and the polymer chains aggregate to cause turbidity. Conversely, at temperatures of 31° C. and below, the polymer chains hydrate to become dissolved in water, thereby causing release of the cell sheet from the polymer. In a particular embodiment, this polymer covers the surface of a substrate such as a Petri dish and is immobilized on it. Therefore, at temperatures above 31° C., the polymer on the substrate surface also dehydrates but since the polymer chains cover the substrate surface and are immobilized on it, the substrate surface becomes hydrophobic. Conversely, at temperatures of 31° C. and below, the polymer on the substrate surface hydrates but since the polymer chains cover the substrate surface and are immobilized on it, the substrate surface becomes hydrophilic. The hydrophobic surface is an appropriate surface for the adhesion and growth of cells, whereas the hydrophilic surface inhibits the adhesion of cells and the cells are detached simply by cooling the culture solution.

The SG cells (e.g. SMG cells) may be added to the culture solution on the temperature-responsive polymer in the cell culture support at various cell densities to optimize formation of the cell sheet or its characteristics. For example, in some embodiments the initial cell density of the SMG cells in the cell culture support used for preparation of the cell sheet is from 1×10⁵/cm² to 9×10⁶/cm² In some embodiments, the initial cell density of the SMG cells in the cell culture support is at least 1×10⁵, 1.5×10⁵, 2×10⁵, 3×10⁵, 4×10⁵, 5×10⁵, 6×10⁵, 7×10⁵, 8×10⁵, 9×10⁵, 1×10⁶, 1.5×10⁶, 2×10⁶, 3×10⁶, 4×10⁶, 5×10⁶, 6×10⁶, 7×10⁶, 8×10⁶, or 9×10⁶ cells/cm². Any of these values may be used to define a range for the initial cell density of the SMG cells in the cell culture support. For example, in some embodiments, the initial cell density in the cell culture support is from 2×10⁵ to 5×10⁶ cells/cm², 4×10⁵ to 5×10⁶ cells/cm², or 1×10⁵ to 5×10⁶ cells/cm².

The SG cells (e.g. SMG cells) may be cultured on the culture support for at least 4, 5, 6, 7, 8, 9, 10, 11, 12, 13, 14 or 15 days before adjusting the temperature of the culture solution to below the lower critical solution temperature (adjusting step b), and detaching the cell sheet from the culture support (detaching step c). In some embodiments, the adjusting step is performed when the SMGs are confluent.

The SG cell sheet may be prepared in a range of different sizes depending on the application. In some embodiments, the SG cell sheet has a diameter of at least 1, 2, 3, 4, 5, 6, 7, 8, 9, 10, 15 or 20 cm. Any of these values may be used to define a range for the size of the SG cell sheet. For example, in some embodiments, the SG cell sheet has a diameter from 1 to 20 cm, from 1 to 10 cm or from 2 to 10 cm. In some embodiments, the SG cell sheet has an area of at least 1, 2, 3, 4, 5, 6, 7, 8, 9, 10, 15, 20, 30, 40, 50, 60, 70, 80, 90, 100, 150, 200, 250 or 300 cm². Any of these values may be used to define a range for the size of the SG cell sheet. For example, in some embodiments, the SG cell sheet has an area from 1 to 100 cm², 3 to 70 cm², or 1 to 300 cm². The methods described herein result in an SG cell sheet in which the surface area of the cell sheet is much greater than its thickness. For example, in some embodiments the ratio of the surface area of the SG cell sheet to its thickness is at least 10:1, 100:1, 1000:1, or 10,000:1. The SG cell sheets described herein comprise one or more layers of confluent SG cells (e.g. SMG cells), for example, 1, 2, 3, 4, 5, 6, 7, 8, 9 or 10 layers of SG cells (e.g. SMG cells). In some embodiments, the SG cell sheet comprises fewer than 2, 3, 4, 5, 6, 7, 8, 9 or 10 layers of SG cells (e.g. SMG cells). In some embodiments, the SG cell sheet comprises at least 1, 2, 3, 4, 5, 6, 7, 8, 9 or 10 layers of SG cells (e.g. SMG cells).

In some aspects, the disclosure also relates to a cell sheet produced by any of the methods described herein.

IV. Methods of Treatment

The SG cell sheets described herein can be transplanted to a subject by applying the cell sheet to a tissue (e.g. a submandibular gland) in the subject. For example, as disclosed in Example 2 below, when an SMG cell sheet was prepared by the methods described herein and implanted onto a surgically wounded SMG, wound closure was improved and salivary flow rates were increased.

Accordingly, in some aspects, the present disclosure relates to a method of transplanting an SG cell sheet to a subject comprising applying an SG cell sheet as described herein to a tissue of a subject. In a particular embodiment, the subject is a human. A support membrane may be used to transfer the SG cell sheet to the tissue of the subject. The support membrane can be, for example, poly(vinylidene difluoride) (PVDF), cellulose acetate, and cellulose esters. The SG cell sheets readily adhere to target tissue, self-stabilizing without suturing after being placed directly onto the target tissue for a short period of time. For example, in some embodiments, the SG cell sheet adheres to the target tissue within 5, 10, 15, 20, 25, or 30 minutes after contact with the tissue. Once the SG cell sheet has adhered to the target tissue, the support membrane may be excised. In some embodiment, the SG cells are autologous to the subject, i.e. isolated from the same subject to which the cell sheet is applied. In certain embodiments, the SG cells in the cell sheet are allogeneic to the subject, i.e. are isolated from a different individual from the same species as the subject, such that the genes at one or more loci are not identical.

In certain aspects, the present disclosure relates to a method of treating a wound in a salivary gland (SG) (e.g. an SMG), the method comprising applying one or more cell sheets as described herein to a wounded SG (e.g. an SMG). Applying the one or more cell sheets to the wounded SG may result in regeneration of new SG tissue and wound closure. In some embodiments, applying the one or more cell sheets to the wounded SG results in wound closure of at least 10%, 20%, 30%, 40%, 50%, 60%, 70%, 80%, 90%, 95% or 99%. Expression of various marker proteins may be measured in the wounded SG to determine whether the newly formed tissue displays polarity and differentiation. For example, in some embodiments, applying the cell sheet or the composition to the wounded SG increases expression of one or more proteins selected from the group consisting of zonula occludens-1 (ZO-1), E-cadherin, aquaporin 5 (AQP5), cytokeratin 7, transmembrane protein 16 (TMEM16), and sodium potassium ATPase (Na⁺/K⁺-ATPase) in the wounded SG and/or the newly regenerated SG tissue relative to a wounded SG to which the cell sheet or composition is not applied.

In certain aspects, the present disclosure relates to a method of treating hyposalivation in a subject, the method comprising applying one or more SG cell sheets (e.g. SMG cells sheets) as disclosed herein to a salivary gland (SG) (e.g. a submandibular gland) in a subject. The term “hyposalivation” as used herein refers to a reduction in saliva production, flow, and/or volume as compared to normal saliva production, flow and/or volume generally found in a healthy subject. Hyposalivation may be due to various causes including, but not limited to, medication, radiation treatment, or an autoimmune disease (e.g., Sjogren's syndrome).

In a particular aspect, the present disclosure relates to a method of treating irradiation damage in a salivary gland (SG) (e.g. an SMG), the method comprising applying one or more SG cell sheets (e.g. SMG cell sheets) as disclosed herein to an SG (e.g. an SMG) that has been damaged by irradiation.

Applying one or more SG cell sheets as disclosed herein to the SG may increase saliva flow rate in the subject relative to a subject in which the one or more SG cell sheets is not applied. In some embodiments, the saliva flow rate is increased by at least 10%, 20%, 30%, 40%, 50%, 60%, 70%, 80%, 90%, 100%, 200%, 300%, 400% or 500% in the subject to which the cell sheet or composition is applied relative to a subject in which the cell sheet or composition is not applied.

Applying one or more SG cell sheets as disclosed herein to the SG may also improve the quality of the saliva relative to a subject (e.g. a subject having a wounded or irradiated SG) in which the one or more SG cell sheets is not applied. For example, in some embodiments, applying the one or more SG cell sheets improves saliva protein composition, e.g. resulting in a saliva protein composition that is the same as or similar to a healthy (e.g. unwounded or non-irradiated) SG in a control subject. Specifically, in some embodiments, applying one or more SG cell sheets as disclosed herein to the SG increases levels of proline rich protein and/or cystatin relative to a subject in which the one or more SG cell sheets is not applied. In some embodiments, applying one or more SG cell sheets as disclosed herein to the SG increases levels of proline rich protein and/or cystatin to a level that the same as or similar to a healthy (e.g. unwounded or non-irradiated) SG in a control subject.

In some embodiments at least 2, 3, 4, 5, 6, 7, 8, 9 or 10 SG cell sheets are applied to the SG in the subject. In a particular embodiment, two SG cell sheets are applied to the SG in the subject. As discussed above, the two or more cell sheets may be stacked on top of each other and cultured for one or more days to allow for further differentiation of the cell sheets before transplantation to the subject.

In particular embodiments of the aforementioned methods, the subject is a human.

### EXAMPLES

### Example 1. Preparation and In Vitro Characterization of Mouse Submandibular Gland (SMG) Cell Sheets

Methods

Animals

Female C57BL/6J mice 6-weeks-old, weighing approximately 17-20 g, were purchased from the Jackson Laboratory (Bar Harbor, Me.). All animal usage, anesthesia, and surgeries were conducted with the approval of the University of Utah Institutional Animal Care and Use Committee, in accordance with their strict guidelines.

Fresh SMG Cell Isolation

Mice were euthanized using 80-100 mg/kg Ketamine+5 mg/kg Xylazine followed by abdominal exsanguination. SMG were then removed, cut into small pieces and placed in a 35 ml GentleMACS™ C Tube containing 6.5% tumor dissociation enzyme mixture (Miltenyi Biotec Inc. Auburn, Calif.) in DMEM/F12 (Invitrogen, Carlsbad, Calif.). Subsequently, the tissue was dissociated using a GentleMACS (Miltenyi Biotec Inc) and incubated in a shaking water bath at 37° C. for 30 min. After three such steps and two intervening incubations, SMG cells were centrifuged at 150×g for 5 min at 4° C. and the dispersion medium was removed. The cells were then resuspended in 5 ml DMEM/F12 complete medium containing the following: 2.5% fetal bovine serum (FBS), 2 nM triiodothyronine, 0.1 μM retinoic acid, 0.4 μg/ml hydrocortisone, 80 ng/ml epidermal growth factor (EGF), 5 ng/ml sodium selenite, 5 mM glutamine, 5 μg/ml insulin and 5 μg/ml transferrin. Cells were then passed through 70 μm and 40 μm strainers (Thermo Fisher Scientific, Waltham, Mass.) and seeded at 1.0×10⁶ cells/plate (1.0×10⁵ cells/cm²) on FBS coated 35-mm UpCell™ temperature-responsive dishes (Thermo Fisher Scientific), cultured at 37° C. in a humidified atmosphere of 95% air-5% CO₂ and used at confluence (a time when monolayer completely covers the plate), with the cell culture medium replaced every other day.

Cell Sheet Preparation

After 8 days of incubation, temperature was reduced below 25° C. and cell sheet was detached from the dish surface within 30 min. After removing culture medium from single layered cell sheet, a wet transfer membrane was placed over the SMG monolayer and 20 μl of fresh medium was added to prevent the cells from drying out. After 30 min of incubation at room temperature, the attached cell layer was transferred to a new FBS coated culture dish and incubated at 37° C. for 30 min. Then, one milliliter of fresh medium was added on top of the membrane and gently removed from the cell layer. After aspirating the culture medium, the first cell sheet layer was covered with a second cell sheet attached membrane, and incubated at 37° C. for 30 min. Finally, the membrane was gently withdrawn from the cell layers and a double layer cell sheet was cultured for one day for further experiments.

Hematoxylin and Eosin Staining

Cell sheets were fixed in 4% PFA for 10 min and fixed in 10% formalin at room temperature for 1 day. Specimens were dehydrated in serial ethanol solutions, embedded in paraffin wax and cut into 3 μm sections. Then, they were deparaffinized with xylene and rehydrated with serial ethanol solutions and distilled water. Finally, hematoxylin and eosin staining was performed, and specimens were examined using a Leica DMI6000B inverted microscope (Leica Microsystems, Wetzlar, Germany).

Transmission Electron Microscopy

Cells sheets or tissues were fixed overnight at 4° C. in a solution containing 2.5% glutaraldehyde, 1% paraformaldehyde, 100 mM cacodylate buffer at pH 7.4, 6 mM CaCl2, and 4.8% sucrose. The next day, cells were washed three times for 5 min each with cacodylate buffer, post-fixed with 2% osmium tetroxide at room temperature for 45 min, washed twice for 5 min with cacodylate buffer, then washed once with distilled water for 5 min. Specimens were then stained with saturated uranyl acetate for 45 min at room temperature, washed 3 times for 5 min each with distilled water, then dehydrated with consecutive ethanol washes (30%, 50%, 70%, twice at 95%, and three times with 100%) for 15 min each. This was followed by dehydration with absolute acetone three times for 10 min each. Specimens were infiltrated with consecutive EPON epoxy resin incubations (30% for 5 h, 70% overnight, three times with 100% for 8 h). 70 nm thick sections were made using a Leica Ultra Cut 6 ultratome, and imaged using a JEOL JEM-2800 operated at an accelerating voltage of 200 kV.

Confocal Analysis

A detailed procedure of deparaffinization and antigen retrieval methods of 3 μm thick paraffin embedded samples can be obtained from a previous study (Nam et al., J Dent Res. 2017; 96:798-806). Specimens were then blocked in 5% goat serum in PBS for 1 h at room temperature, and incubated at 4° C. with the primary antibodies in 5% goat serum overnight as follows: rabbit anti-ZO-1 (Invitrogen, 1:50 dilution), mouse anti-E-cadherin (BD Biosciences, San Jose, Calif., 1:100 dilution), rabbit anti-aquaporin 5 (Abcam, Cambridge, Mass. 1:200 dilution), mouse anti-cytokeratin 7 (Abcam, 1:250 dilution), rabbit anti-TMEM16A (Abcam, 1:50 dilution) and mouse anti-Na⁺/K⁺-ATPase a antibody (Santa Cruz Biotechnology, Santa Cruz, Calif. 1:100 dilution). Then, sections were incubated for 2 h with anti-rabbit Alexa Fluor 488 and anti-mouse Alexa Fluor 568 secondary antibody solution at 1:200 dilutions in 5% goat serum at room temperature. Subsequently, specimens were counter-stained with TO-PRO-3 Iodide nuclear stain (Invitrogen) at room temperature for 15 min at 1:1000 dilutions. Finally, specimens were analyzed using a confocal Zeiss LSM 700 microscope (Carl Zeiss, Oberkochen, Germany) at 20× magnifications for in vivo studies and 40× for in vitro studies. A total depth of 3 μm was acquired for each sample, and a total projection was visualized in the xy planes.

Results

Single Layer Cell Sheet Formation

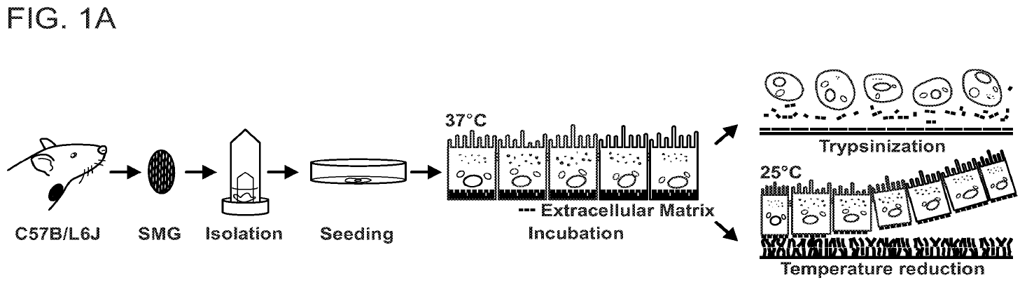

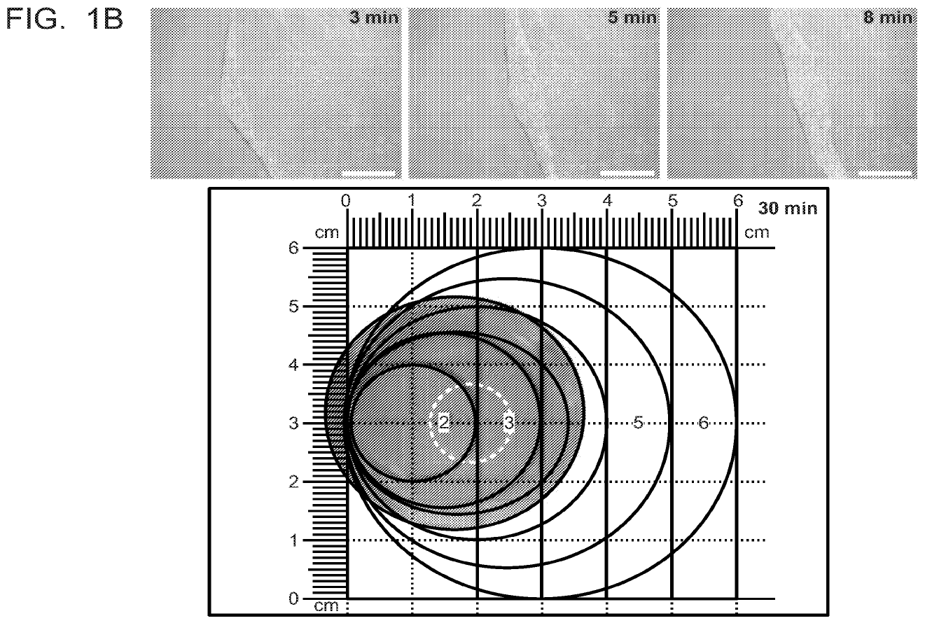

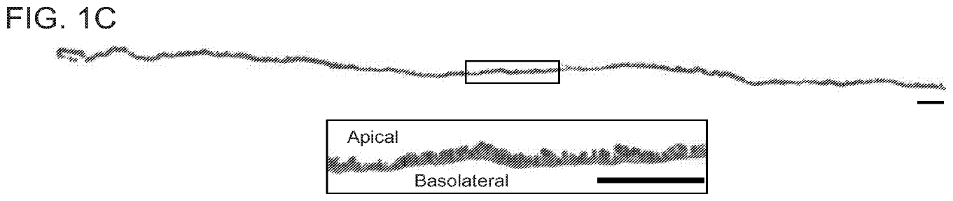

To investigate whether freshly isolated mouse SMG cells were able to form a cell sheet, cells were cultured on a polystyrene dish covalently covered with a temperature-responsive polymer PNIPAAm (i.e., thermoresponsive cell culture dish) for eight days as described above and depicted in FIG. 1A. Our results show that cells formed a single layer capable of detaching from the culture dish when the temperature is decreased from 37° C. to 25° C. (FIG. 1B). A single layer cell sheet (FIG. 1C) display a closely packed columnar pattern with a flat basolateral side and a protrusive apical side (FIG. 1C). Together, these results demonstrate that SMG are capable of forming polarized cell sheets in vitro.

Transmission Electron Microscopy Studies

Since SMG cells formed single layer sheets, we determined whether they displayed a polarized secretory phenotype. As shown in FIG. 2A, single cell sheets were able to form structures consistent with intercellular junctions including tight junctions (TJ), adherens junctions (AJ) as well as desmosomes (DS). Additionally, we detected microvilli-like structures on the apical side of the cell sheet (FIG. 2A) and secretory granules (SG) located towards the apical membrane (FIG. 2B) which was similar with a C57BL/6J mouse native SMG specimen (FIG. 2C). Thus, a SMG-derived single layer cell sheet has features consistent with polarized secretory epithelia.

Confocal In Vitro Studies

To confirm the presence of TJ proteins and detect markers of SMG differentiation, we analyzed cell sheet sections by confocal microscopy. Our results showed that single layer cell sheets display a columnar epithelial-like arrangement (FIG. 3A) expressing epithelial junctions including the apical TJ protein zonula occludens-1 (ZO-1, FIG. 3B, green) as well as the basolateral protein E-cadherin (FIG. 3B). Moreover, single layer cell sheets expressed the salivary gland acinar marker aquaporin 5 (AQP5, FIG. 3C) with F-actin (FIG. 3C). These results suggest that SMG cells are capable of forming single layer sheets that polarize and differentiate but do not display the typical three-dimensional glandular-like arrangements of a salivary gland.

Differentiation of Cell Sheets In Vitro

Since single cell sheets did not form a glandular appearance, we investigated whether a combination of two single layer cell sheets promotes formation of a glandular-like appearance tissue in vivo. As shown in FIG. 3D-F, placing two single cell sheets on top of each other for one day, formed a double layer cell sheet with a glandular-like appearance where the majority of cells displayed organized round structures consistent with epithelial lumens. Together, these results indicate that double layer cell sheets display a more organized pattern as compared to single cell sheets and therefore may have more advantageous properties for use in vivo.

These results demonstrate that some of the advantages of SMG cell sheets for in vitro studies is that cells grown under these conditions can be cultured as monolayers while maintaining all intrinsic ECM proteins, TJs and secretory granules (FIG. 2). Additionally, double layer cell sheets are able to form acinar and ductal-like organoids with a three-dimensional shape containing lumens and consistent with salivary gland epithelium (FIG. 3). These features offer an improvement over freshly isolated SMG cells plated on plastic where they lose secretory granules, form disorganized TJs and de-differentiate over time (Redman et al., Biotech Histochem. 2008; 83:103-30). For use of in vitro models, one could measure salivary gland cells' ability to form cell sheets under conditions such as pro-inflammatory cytokine exposure or irradiation, perform phosphorylation and protein expression studies (given that these organoids are similar to native tissue) and/or detect intracellular calcium signaling in response to secretory agonists (Maruyama et al., J Dent Res. 2015; 94:1610-7). Likewise, each of these functions could in turn be measured in cell sheets derived from human SMG, given that such tissue is relatively easy to obtain and culture (Maruyama et al., Comparing human and mouse salivary glands: A practice guide for salivary researchers. Oral Dis. 2018). Together, these studies demonstrate that cell sheets could be used as a good in vitro model for studying salivary gland cell signaling and function.

### Example 2. In Vivo Characterization of Mouse Submandibular Gland (SMG) Cell Sheets

Methods

Animal Model

The wounded SMG model was created following a method reported previously (Nam et al., J Dent Res. 2017; 96:798-806; Nam et al., PLoS ONE. 2017; 12:e0187069). Briefly, C57BL/6J mice were anesthetized with 3% isoflurane with an oxygen flow rate set at 2.0 L/min, SMGs were exposed and surgical wounds created using a 3 mm diameter biopsy punch. The treatment groups were as follows: (1) wounded and treated with a single layer cell sheet (experimental group SC), (2) wounded and treated with a double layer cell sheet (experimental group DC), (3) untreated wounded control or (4) unwounded (sham surgery controls). After surgery, the skin incision was sutured, and post-surgical studies were performed at day 8 or 20. For these purposes, SMG were dissected and processed for histological analysis and saliva secretion studies as described below.

Saliva Flow Rate Measurements

To collect stimulated saliva secretion, mice were anesthetized with ketamine (100 mg/kg) and xylazine (5 mg/kg), and injected with pilocarpine (50 mg/kg) and isoproterenol (0.5 mg/kg) via intraperitoneal injection. Then, stimulated saliva was collected using a micropipette for 5 min (Nam et al., PLoS ONE. 2017; 12:e0187069). Finally, statistical significance was assessed by one-way ANOVA (P<0.05) and Dunnett's post hoc test for multiple comparisons to the untreated group and sham control group.

Weight Change

Mice were weighed at the start of each experiment and data was collected for 8 days. Then, statistical significance was assessed by two-way ANOVA (P<0.05) and Dunnett's post-hoc test for multiple comparisons to the untreated group.

Results

Effects of Double Layer Cell Sheets In Vivo

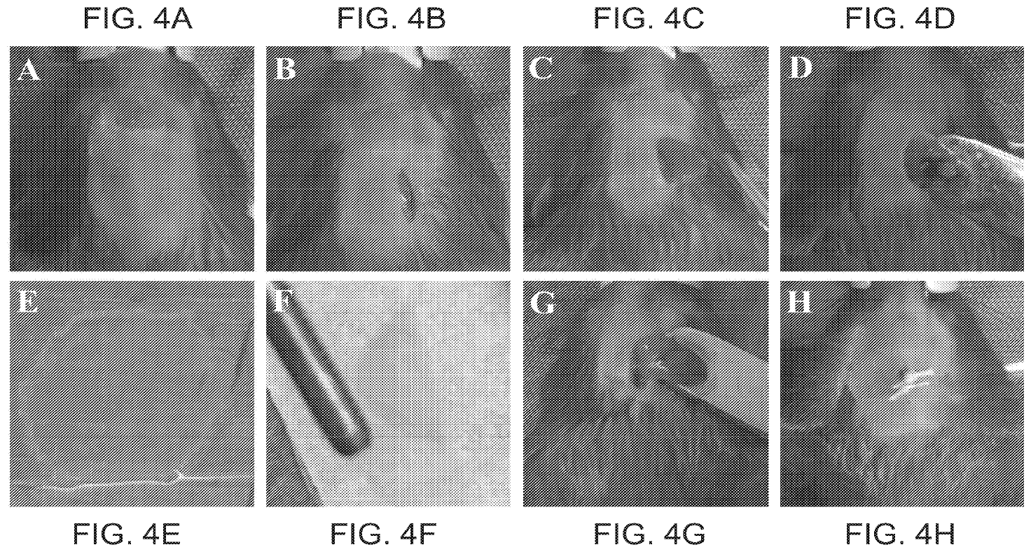

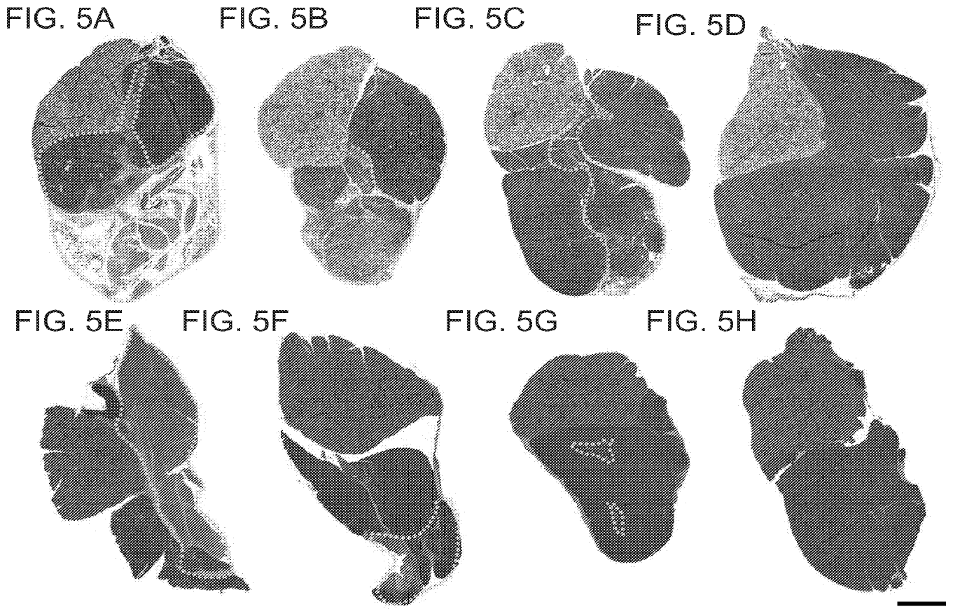

Given that double layer cell sheets were able to form a glandular-like appearance in vitro, we decided to use double layer cell sheets for regeneration studies in vivo by transplanting SMG double layer cell sheets into a wounded mouse model (FIG. 4), as described above. Our results indicate that untreated SMG surgical wounds displayed fibrotic tissue at post-surgery day 8 (FIG. 5A). In contrast, SMG surgical wounds transplanted with a double layer cell sheet (FIG. 5C) displayed an approximately 90% wound closure with a similar morphology to unwounded controls (FIG. 5D). Together, the histological results demonstrate that a double layer cell sheet promotes regeneration in wounded mouse SMG after eight days.

Confocal In Vivo Studies

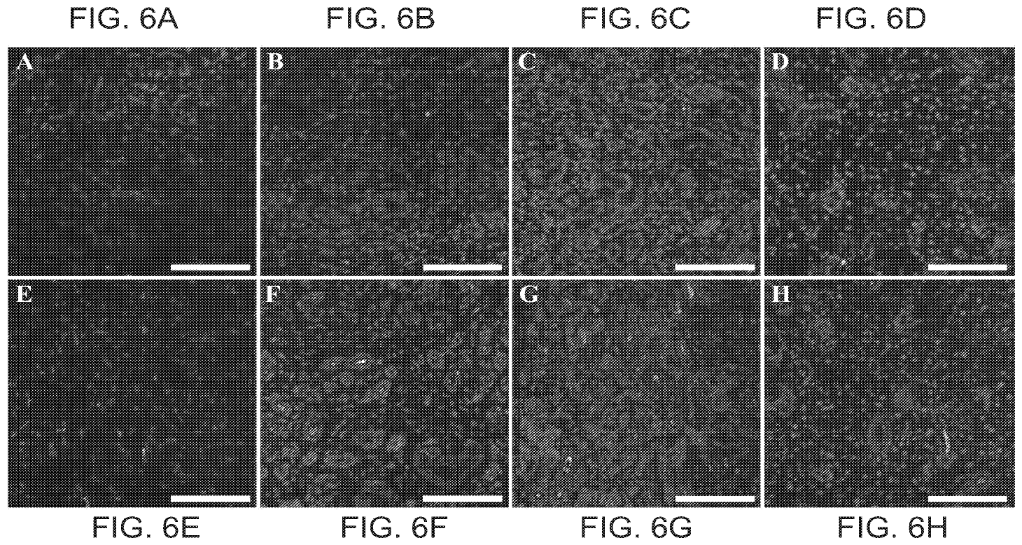

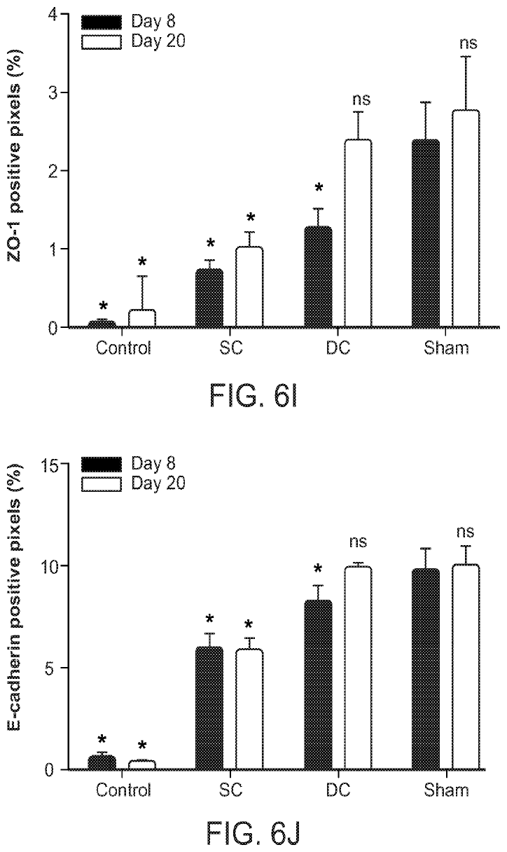

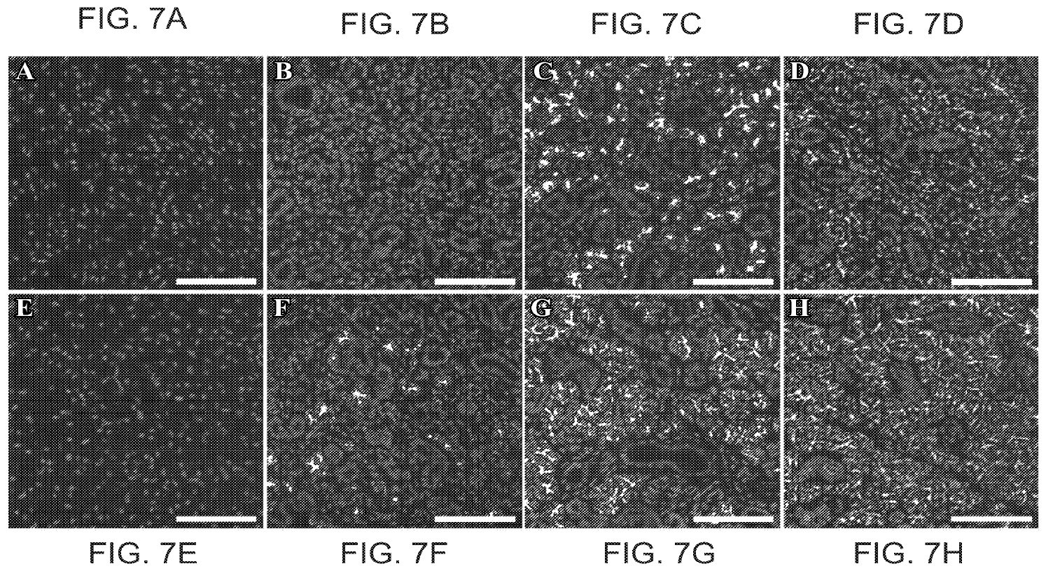

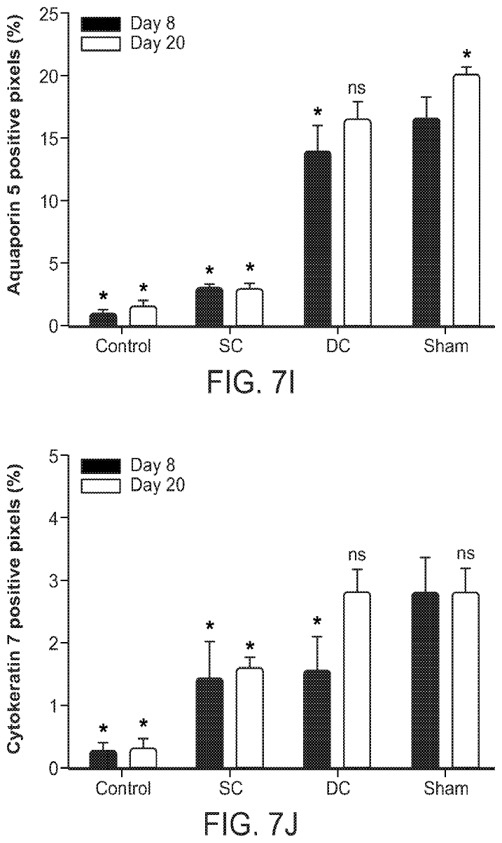

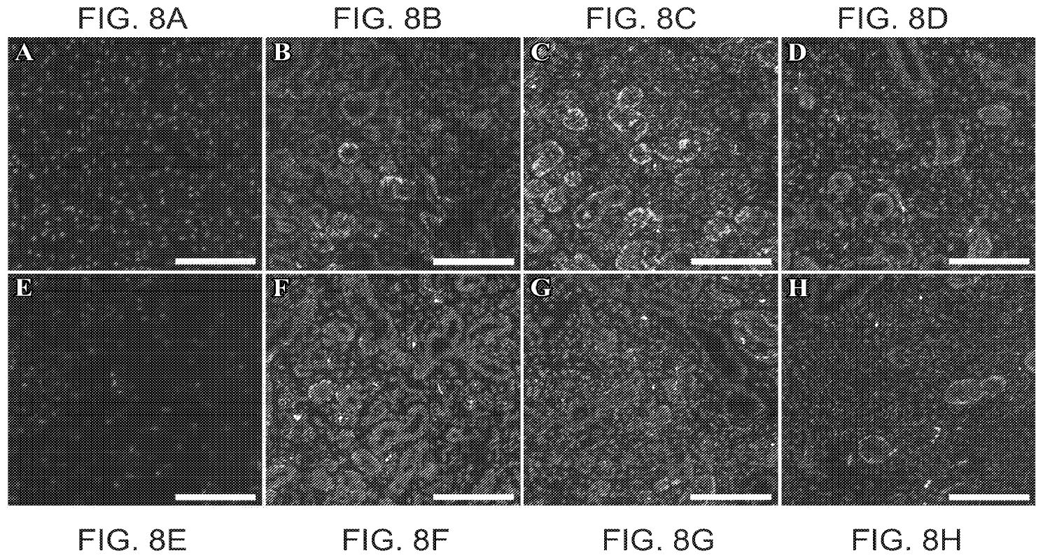

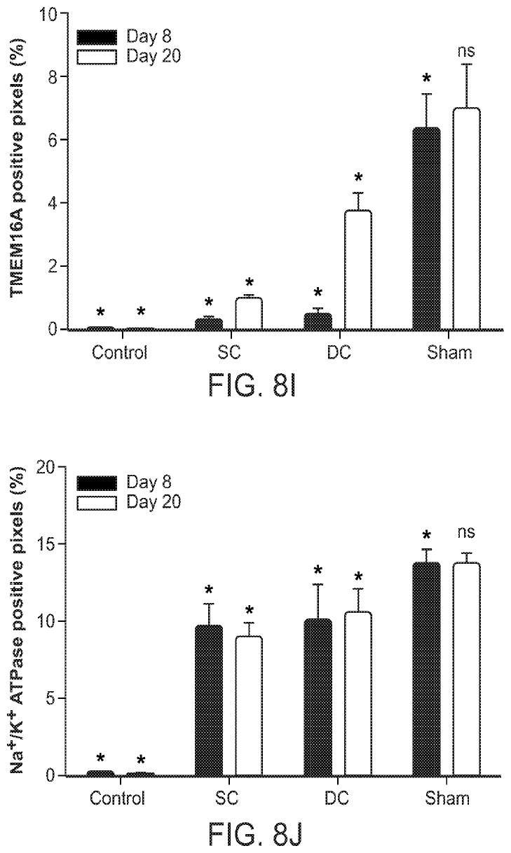

Since a double layer cell sheet promotes regeneration in wounded mouse SMG, we determined whether the newly formed tissue displayed polarity and differentiation under these conditions. Our results show that untreated wounded SMG expressed both ZO-1 (FIGS. 6A and E) and E-cadherin (FIGS. 6A and E), with either protein expressing only a very weak staining together with a disorganized structure. In contrast, wounded SMG transplanted with a double layer cell sheet expressed ZO-1 (FIGS. 6C and G) and E-cadherin (FIGS. 6C and G) with a strong apical and basolateral staining respectively, similar to sham controls (FIGS. 6D and H) and indicating polarity. Moreover, untreated wounded SMG barely expressed the acinar marker aquaporin 5 (AQP5) (FIGS. 7A, 7E and 7I) or the ductal marker cytokeratin 7 (FIGS. 7A, 7E and 7J), with both proteins showing a weak staining and a disorganized pattern. On the contrary, wounded SMG transplanted with a double layer cell sheet expressed AQP5 (FIGS. 7C, 7G, and 7I) and cytokeratin-7 (FIGS. 7C, 7G and 7J), where both proteins displayed strong apical and basolateral staining patterns, respectively, similar to unwounded controls (FIGS. 7D and H) and indicating differentiation. Finally, untreated wounded SMG barely express the acinar marker transmembrane protein 16 (TMEM16, FIGS. 8A, 8E and 8I) or the functional basolateral marker sodium potassium ATPase (Na⁺/K⁺-ATPase, FIGS. 8A, 8E and 8J), with both proteins showing a weak staining and a disorganized pattern. Conversely, wounded SMG transplanted with a double layer cell sheet expressed weak apical TMEM16 (FIGS. 8C, 8G and 8I) and strong basolateral Na⁺/K⁺-ATPase (FIGS. 8C, 8G and 8J), similar to unwounded controls (FIGS. 8D and H) and indicating functionality.

Body Weight and Saliva Secretion Studies

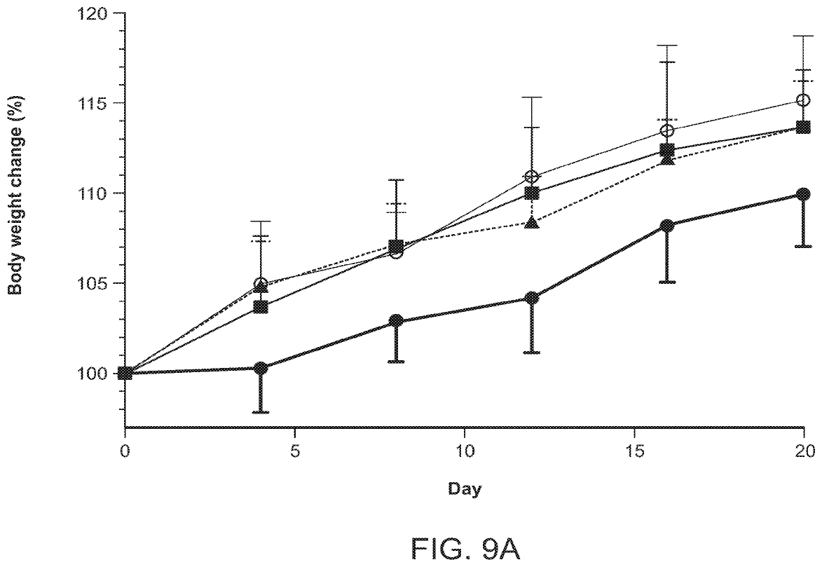

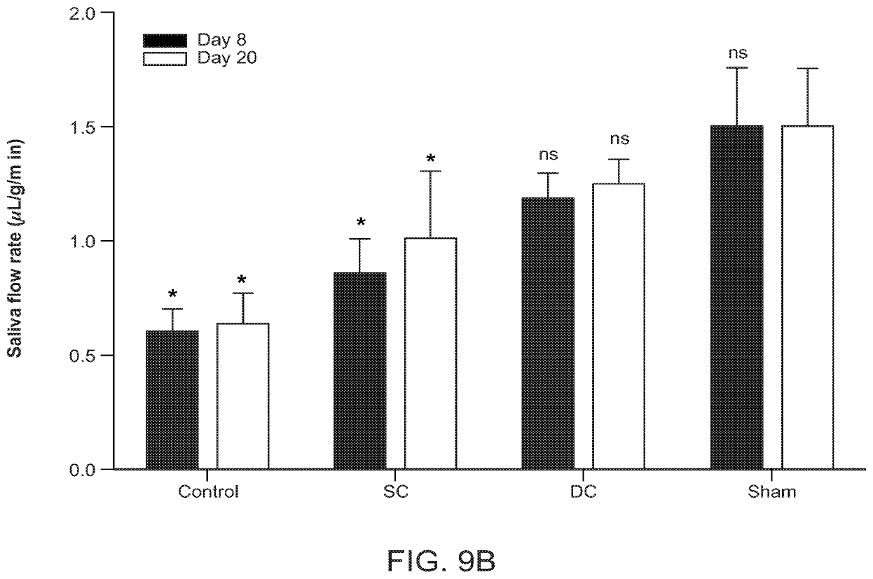

Our previous studies indicated a decrease in saliva flow rates in wounded mouse SMG (Nam et al., J Dent Res. 2017; 96:798-806; Nam et al., PLoS ONE. 2017; 12:e0187069. Since saliva is critical for eating and swallowing, we determined the capability of the mice to perform these functions by measuring their body weight at various post-surgery times. Our results show that untreated wounded mice exhibited a significant decrease in body weight (FIG. 9A). In contrast, wounded mice treated with a double layer cell sheet displayed body weights comparable to sham controls (FIG. 9A). Since a double layer cell sheet promoted regeneration in wounded mouse SMG as compared to controls, we determined whether the newly formed tissue improved salivary secretory function under these conditions. Our results show that untreated wounded mice exhibited a significant decrease in saliva flow rates (FIG. 9B). In contrast, wounded mice treated with a double layer cell sheet exhibited saliva flow rates comparable to unwounded controls (FIG. 9B).

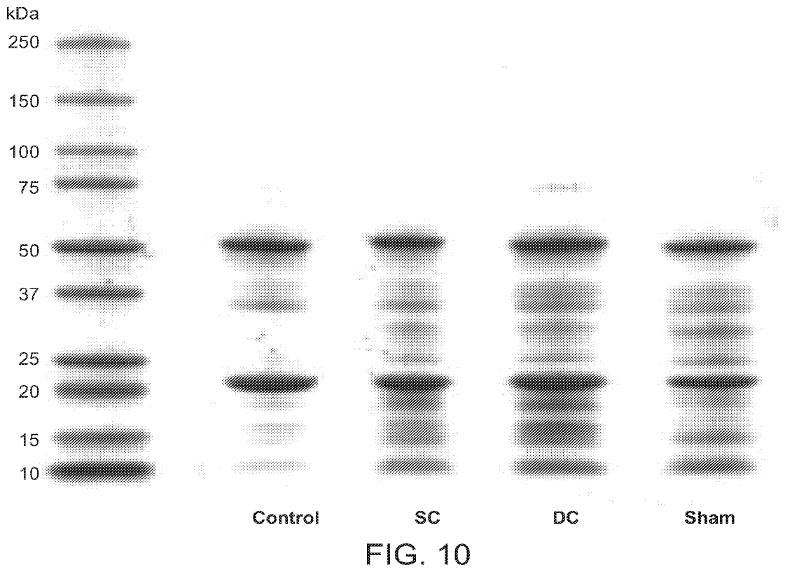

To determine the saliva composition of each treatment group, 15 μg of saliva protein from each group was fractionated by SDS-PAGE. Saliva samples were denatured at 95° C. for 5 min in a sample loading buffer. The denatured samples were loaded onto the Mini-PROTEAN TGX precast electrophoresis gel and subjected to electrophoresis in 25 mM Tris/192 mM Glycine buffer with 0.1% SDS (w/v) at 100 V for 70 min. The electrophoresis gel was fixed in a solution of 25% ethanol, 15% formaldehyde, 60% water and stained with 0.25% Coomassie Brilliant Blue R-250 in 50% (v/v) methanol, 10% (v/v) glacial acetic acid for 1 h and destained overnight in 20% (v/v) methanol and 10% (v/v) acetic acid. Protein images of gels were captured using a Chemi Docmp imaging system (Bio-Rad). As shown in FIG. 10, the total protein composition of the saliva from untreated wounded mice (control) showed clearly different patterns compared to the saliva from the untreated, unwounded (sham) control group, indicating that wounding affects saliva protein composition. For example, the untreated, wounded control group displayed decreased proline rich protein (15 kDa˜30 kDa) and cystatin (10 kDa) levels relative to the unwounded (sham) control group. However, the protein patterns of the cell sheet treated groups (SC and DC) showed comparable protein patterns to sham control (FIG. 10). These results indicate that the cell sheet treated wounded SMG could produce a similar quality of saliva as compared to unwounded (sham) controls.

Regarding the advantages of cell sheets for in vivo studies, autologous cells could be used for transplantation, as cells sheets completely detach from temperature-responsive plate without carrying any chemicals or contaminants. Moreover, because cell sheets are able to maintain ECM proteins, they can rapidly attach to target organ surfaces without needing sutures, thereby facilitating regenerative treatments. See Akimoto et al., Anticancer Res. 2018; 38:671-6; Akiyama et al., Biomacromolecules. 2018; 19:4014-22; Sekine et al., Cell Sheet Tissue Engineering for Heart Failure. In: Nakanishi T, Markwald R R, Baldwin H S, Keller B B, Srivastava D, Yamagishi H, editors. Etiology and Morphogenesis of Congenital Heart Disease: From Gene Function and Cellular Interaction to Morphology. Tokyo 2016. p. 19-24; Takahashi et al., Sci Rep. 2018; 8:13932; Yamaguchi et al., Sci Rep. 2017; 7:17460; Yamato et al., Materials Today. 2004; 7:42-7. In this regard, the results described above showed that a double layer cell sheet optimizes tissue formation (FIG. 5), cell differentiation (FIG. 6, 7, 8) and quantity (FIG. 9) and quality (FIG. 10) of saliva. In contrast, untreated mice did not show these improvements.

### Example 3. Preparation of Human Submandibular Gland (SMG) Cell Sheets

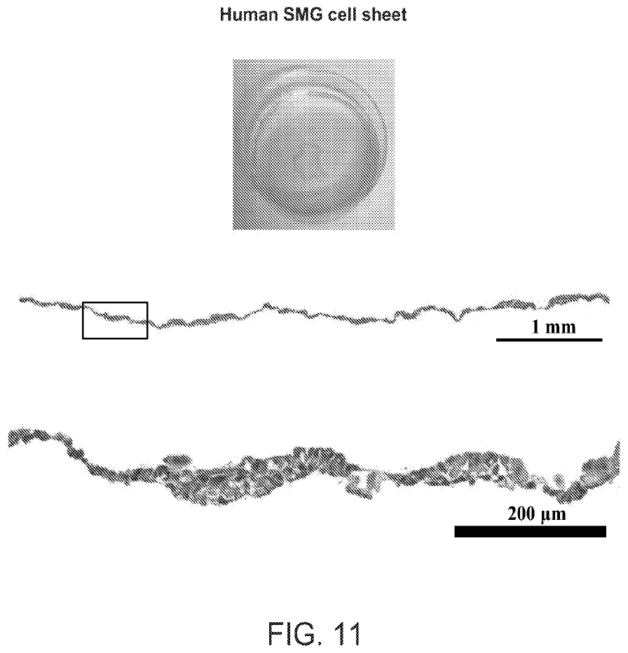

To isolate human SMG cells for preparation of cell sheets, human SMG tissue (about 100 mg) was cut into small pieces and placed in a 35 ml GentleMACS™ C Tube containing 6.5% tumor dissociation enzyme mixture (Miltenyi Biotec Inc. Auburn, Calif.) in DMEM/F12 (Invitrogen, Carlsbad, Calif.). The tissue was dissociated using a GentleMACS dissociator and incubated in a shaking water bath at 37° C. for 30 min. After three dissociation steps and two incubation steps, SMG cells were centrifuged at 150×g for 5 min at 4° C. and the medium was removed. The cells were then resuspended in 5 ml DMEM/F12 complete medium containing the following: 2.5% FBS, 2 nM triiodothyronine, 0.1 μM retinoic acid, 0.4 μg/ml hydrocortisone, 80 ng/ml EGF, 5 ng/ml sodium selenite, 5 mM glutamine, 5 μg/ml insulin and 5 μg/ml transferrin, and passed through 70 μm and 40 μm strainers (Thermo Fisher Scientific, Waltham, Mass.). Then, 1.5×10⁶ cells were seeded on FBS coated 35-mm UpCell™ temperature-responsive dishes (Thermo Fisher Scientific), and cultured at 37° C. in a humidified atmosphere of 95% air-5% CO₂ for 8 days. The human SMG cell sheets are shown in FIG. 11.

We claim:

1. A salivary gland (SG) cell sheet comprising one or more layers of confluent SG cells.

2. The cell sheet of claim 1, wherein the SG cells are arranged in a columnar pattern with a flat basolateral side and a protrusive apical side.

3. The cell sheet of claim 1, wherein the cell sheet comprises one or more intercellular junctions selected from the group consisting of tight junctions (TJ), adherens junctions (AJ), and desmosomes (DS).

4. The cell sheet of claim 1, wherein at least 50% of the SG cells in the cell sheet are connected by an intercellular junction selected from the group consisting of tight junctions (TJ), adherens junctions (AJ) and desmosomes (DS).

5. The cell sheet of claim 1, wherein the cell sheet comprises one or more of microvilli-like structures and secretory granules.

6. The cell sheet of claim 1, wherein at least 50% of the SMG cells in the cell sheet comprise microvilli-like structures or secretory granules.

7. The cell sheet of claim 1, wherein the SMG cells express one or more proteins selected from the group consisting of tight junction protein zonula occludens-1 (ZO-1), E-cadherin, aquaporin 5 (AQP5) and F-actin.

8. The cell sheet of claim 1, wherein the cell sheet consists essentially of SG cells.

9. The cell sheet of claim 1, wherein at least 50% of cells in the cell sheet are SG cells.

10. The cell sheet of claim 1, wherein the SG cells are human SG cells.

11. A composition comprising the cell sheet of claim 1 and a polymer-coated culture support that is removable from the cell sheet.

12. A composition comprising at least two of the cell sheet of claim 1.

13. The composition of claim 12, wherein the at least two cell sheets are stacked on top of each other.

14. The composition of claim 13, wherein the cell sheets comprise epithelial lumens.

15. The cell sheet of claim 1, wherein the SG cells are submandibular gland (SMG) cells.

16. A method of treating a wound in a salivary gland (SG), the method comprising applying the cell sheet of claim 1 to a wounded SG.

17-18. (canceled)

19. A method of treating hyposalivation in a subject, the method comprising applying the cell sheet of claim 1 to a salivary gland (SG) in a subject.

20-21. (canceled)

22. A method of treating irradiation damage in a salivary gland (SG), the method comprising applying the cell sheet of claim 1 to a SG that has been damaged by irradiation.

23-26. (canceled)

27. A method for producing a salivary gland (SG) cell sheet comprising one or more layers of confluent SG cells, the method comprising:
a) culturing SG cells in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the culture support.

28-32. (canceled)

33. A cell sheet produced by the method of claim 27.
